SEQUENCE LISTING (15 sequences)

SEQ ID NO: 1 - protein, 1132 aa, Homo sapiens
MGMACLTMTEMEGTSTSSIYQNGDISGNANSMKQIDPVLQVYLYHSLGKSEADYLTFPSGEYVAEEICIAASKACGITPVYHNMFALMSETERIWYPPNHVFHIDESTRHNVLYRIRFYFPRWYCSGSNRAYRHGISRGAEAPLLDDFVMSYLFAQWRHDFVHGWIKVPVTHETQEECLGMAVLDMMRIAKENDQTPLAIYNSISYKTFLPKCIRAKIQDYHILTRKRIRYRFRRFIQQFSQCKATARNLKLKYLINLETLQSAFYTEKFEVKEPGSGPSGEEIFATIIITGNGGIQWSRGKHKESETLTEQDLQLYCDFPNIIDVSIKQANQEGSNESRVVTIHKQDGKNLEIELSSLREALSFVSLIDGYYRLTADAHHYLCKEVAPPAVLENIQSNCHGPISMDFAISKLKKAGNQTGLYVLRCSPKDFNKYFLTFAVERENVIEYKHCLITKNENEEYNLSGTKKNFSSLKDLLNCYQMETVRSDNIIFQFTKCCPPKPKDKSNLLVFRTNGVSDVPTSPTLQRPTHMNQMVFHKIRNEDLIFNESLGQGTFTKIFKGVRREVGDYGQLHETEVLLKVLDKAHRNYSESFFEAASMMSKLSHKHLVLNYGVCVCGDENILVQEFVKFGSLDTYLKKNKNCINILWKLEVAKQLAWAMHFLEENTLIHGNVCAKNILLIREEDRKTGNPPFIKLSDPGISITVLPKDILQERIPWVPPECIENPKNLNLATDKWSFGTTLWEICSGGDKPLSALDSQRKLQFYEDRHQLPAPKWAELANLINNCMDYEPDFRPSFRAIIRDLNSLFTPDYELLTENDMLPNMRIGALGFSGAFEDRDPTQFEERHLKFLQQLGKGNFGSVEMCRYDPLQDNTGEVVAVKKLQHSTEEHLRDFEREIEILKSLQHDNIVKYKGVCYSAGRRNLKLIMEYLPYGSLRDYLQKHKERIDHIKLLQYTSQICKGMEYLGTKRYIHRDLATRNILVENENRVKIGDFGLTKVLPQDKEYYKVKEPGESPIFWYAPESLTESKFSVASDVWSFGVVLYELFTYIEKSKSPPAEFMRMIGNDKQGQMIVFHLIELLKNNGRLPRPDGCPDEIYMIMTECWNNNVNQRPSFRDLALRVDQIRDNMAG

SEQ ID NO: 2 - DNA, 19 bp, Homo sapiens
  source 1..19: mol_type = genomic DNA
tttccttagtctttctttg

SEQ ID NO: 3 - DNA, 16 bp, Homo sapiens
  source 1..16: mol_type = genomic DNA
ccttagtctttctttg

SEQ ID NO: 4 - RNA, 19 nt, synthetic construct
  misc_feature 1..19: note = antisense oligonucleotide (ASO)
  source 1..19: mol_type = other RNA
caaagaaagactaaggaaa

SEQ ID NO: 5 - RNA, 16 nt, synthetic construct
  misc_feature 1..16: note = antisense oligonucleotide (ASO)
  source 1..16: mol_type = other RNA
caaagaaagactaagg

SEQ ID NO: 6 - DNA, 43751 bp, Homo sapiens
  source 1..43751: mol_type = genomic DNA
gatctcagctcactgcaacctctgcctccagggttccagcgattctcctgcctcagtcaccttagtagctgggattacaggcacatgccaccacatctggctaatttttgtatttttagtagagatggggtttcgccacattggccaggctggtctcgaactcctggcctcaagtgatccgcccacctcggcctcccaaaattctgggattacaggcatgagccaccgtgcccagcccaagatcttcttcatttcctaactctcccctgcctgctcagcctcaggtaggtggaggggagaggaagggaggaaagaaagaggggggcctagattcaaaaggaggtggggtctttctttctgtagtctacaaagagggagggtaggtagatcccaaaccgaattattcagaaccgtttttaagcttttattgctgctgccactgcaaagtaaaatttcagtctttctactccagggttgcattaggaggaagaaaacgacttgctccatatgcaaaatgcaccggcctccacaccaaagaccaggcccacccgtcctgttggctggcgcgcaggaggggctccacaccaatctgggagaggaatgaatgctttcccagtggggtagggcttggagctcctagagacatgaactccgtgatctgtggttggtcaatgaccttgagagacaaggaggacaaggaaggtgctgctctctacaaaacgtctttgacctgtttgatgtctagtctccccatacgggttcagcagtgcccagtgtgagaagaacacgctggcaggaaggtgctaacctctcacacaggacacaggggtagtaggcacaacctacccacatgctcacttacaacttcttcctatgctcagaatgttttgccgcttctttgagaagcaagaaagactcccacatctccccactgcagaggatccctggagtcccagcagcagttccagcagccaatgttttgtgagtgctgactgtatgccaggcactgttctaagagctttaaatgtatacatttacttaatcctctccaacacccatgcagatagaaatgaacattacttccatcttgcagaagcctggcgtcccaggctgtgaggggcagccggaactgcaccccgggggggtccctacagtaactgctatccctgcactgctcctgtgtctcccaccagtgagtggaactttgtcgaatgttttctgcagtttatgatacctaaaaatgttttctcaagtataaaattttaacactgaatgtgctttttcaaataatgagtttctcttagagactctgatacaaacttcctcatcagaaatcttgagttaagacttcaagctcctccataataaaagagagcaaacacatacagaatttctctttggtctcttgcagttacgtcttcttttaagggtctcagaaaaataccgtcaaaaaagttaaagctgaaagtggtggtgcatgcctgtaatcccagctactcaggaggctgaggcgggaggaccactttaggccaggagttcaaaaccagtctgggaaacatagtgaaactctgtctcttaaaaacaaaagaggaaatttaaatattattgagaaccaaaaggtaggaagttttgactttagaaagatggttctacaaatgaaaggaaaagaggcaccaaaacccacattttgatattaccctggtagatatgaagtgctctgtgaattgaaccttcgagaagcagaccaaagggaggctgagggaagaagagaaacacagatcattaccattaatactacaaatgcaaactgccctttatgatgctaaatgcacagctgctgctcctggtacatggtatgaaccactgcatgacagcctgcccacagaaacacctaggtgagcaaaactacaagaacgtcacttacagctactggttatatgtgttgagatcagttagccacatcatctacaagcgcctgagaccagaaccacccgtccactaactcatgccttgactaagtgcctggtaggttcagactctgatctgtgcccaagcgtgggagcagcctccaagatggccctgctgggccccgcctcctgctattcttacccctgttagccccctgggagtggcctgtgtgtccaacagctagtgacagacatgctagcacgtggcttctgggatgaggtgataaccaactgggacttccatcttggatgcctgtgctcaccctttctgatctctggccttagggctgccctgctgtgagcagccctacctagaggtccacatggcggggaactaggcctatcgccaacacccatgggaacgaccttgcaggtagattctccagccgctggcgcagtggagtcttgagataactacagcccctgcagacttgcttgctgcaagctaagaccctgagccagaaccagccacttaagctgcttctgtgttcctggctctcagaactggtgtgagttaataaatgttttaagctgctaagttttggggtaatttctgatgtaaccgtagataacgcatacaccaggaatacaccaggaaacaaagacattcaatctactgagaagacaggggatcaaaataagtgcttaggtgtaaaaagcagagaacagtttctcaataaccacttttaaagaaactcagctataatttacacaccataaaaaccacctttttaaagttcacccattagcaccagctccccatttgcatctcctcggctgctttctcctctcctctcccatttgggcatccggggctcaacccttggccttgacctcctctccgcccatggccattcctctggtgactgcatccagtccgatggatttaaacacccccaatgtgctggtgactcccaagtttacacctccagccagggcttctctcctggttttgcatacccacctatctatctgccttagccactcgtgtctgacgagcacctcacacctccagaggctcctcatttcttcccatgcctgcttctcccacactcctccctctcacatgagggcaacttcatcctcccagttgctcaggccccaaacctccatcagttttgactcttctctcgcacactactcagtcaacccattggcaaatccatcccactattaaaaacttagcaggcttctgtacctaatttgcttctagctttgtttttgggtccccagggaacaaacagtctccttcctctcccacaggatttcctttaggtagtcctagagactgtgactgtgtacttccatcacgtctcctttgctcactcttcaaggccccagctgctcactgttttggtcaccttgtctctactcgcttcactgtgtgtctttctctcctaatgcgtgagtctgtacttgaacatagcattggaggggtggtttcatgtaggtaggtagaatggagtaagtcccactgcttttgtaaaaactttcctgcctacttaaacctaagttttattatggaaaatgtcaagcatacaaaagtaaagagaacagaatagtgaactctctcaattatccctttccaggttcaacagttaccaactcatggccaattttggcatctacattgcctctggcccaactctgtgattattattaatttcttaatagactttattttttagcacagttttagtttcacaccaaaattgagtggaaggtacagagagttcccatatgtcccctgtccatgtgcccacagcttcctatcaacatccctcaccagagtggtacaatctttacaattgatgaacctacattgacacatcattgttatccaaagtccatagtctacattaggggttcacccttggtagtgtacattctatggatttggacaaacttataatgacatgtatctaccattatatcatacagaatagtttcactgccctaaaaatcctctgagctccacctatttatccttttctcccccaatcctcttaccccccatgtaactttttactgtctccttagatttgccttttccagaatgtcatagtgggaaacatacaagatgtccccttttcaggttggcttcttttacttagtaacatgtatgtaagtttcctccatgtctcttcatgactcatttctttttattgctgcataatattctactgtatggatgtcctacagtttatgtaaccattcatctcctgaaggacatctgggttgcttccaagttttgggaattatggtactattcttttaatatcgtccaatatagagttagatccagttttccatttttgggggtcatggtgagacatgaaacccaggtttaaaacatacataaacactcatgcacacaaatacacactccaaccattttcaagcagagatttatagagtttttaacggacataacctttttggagcctcgtatcagcccagcgccttaaatacggctgtttcttttgcagaatgagttcagattaggcgaggatatccatcaccatttttcagaccagctcgcagcctgggggcagtggttcaagcacaggctttggagccgaaatgactgcgcctgaagtactcttcttccacttccccgctgtgtgaccctgggcacgttacctagctcctctgagcctcactcttctcccccataacatagggcctataccaaagttcaagggctattgtcagtattcaggctactaactgaaggaatcaaaatctgaacctgggttcccccgactccaaaacccgtgctatattcaccacaaatgcgatcaacacacaaggcaccaggtcggacccaggcacatacgtggcgagaccgcagggagcagcgctcgctctgcgtcctcccttcctcccatatcccaggtctcacctgctccgggccccgaaggtctcaggatgctgtggctgcacgcggacgccagccgccctaaggtgaccgtcagggaagatgctgccatggccgcagcctcggccggaaacggaaacgacgcgaaccgcgtgcgtaacgcacgggcccggcggggaacaccggtccgctgtaacaccggcccggcgcagaagcgggacgtcggcttctaggggcccaggccggcggcggcgatggcctcggcagctgtggagagcttcgtgaccaagcaactggacctgctggagcttgagagagacgcggaggtggaggagcgcaggtacgggaggccgccggcgccgctccctcgcggtcggtcccgccgtgtcccgggcagagtctccgaggggctcggcctcatagtctggggcgggaatttcgtctccgacccttcgagtcaggggcagggatcgtccgtcccctgcttggccacctcccgagttgtcagtctcatcacttcctgggcagcgcgttccgtttggggacccttaagaagcactttgatttcccgacccgagaccccccactaagcttacactcctgggccctggccccgaccccagttccacccgcagcgagttagcagccctttcccttattcattcagtaaacatggtttgcgcccattctcagtcaggccccggcagcatgagtcaggtgcactggctgcccccgatccggccagcggcgggctgggcataagcagagagtcccttcccctccagtgcgtccagggccggctgcgtgcgtgtcgcggggaaagagcctggaggaggaaggcccttccctggggagggtcatggtggcttcaaggagggagtggtgcctgaaggaaaattggagttaagctggtgggtgcggagtagggtggggagccgtcctgagaggtgtggggagccatgtgagagccgggcttgtttccgaatgggactatttacttgtccaaaacaatcagagctaacacttacgtactttccatgcgttaattcttttacacctcatcccatgccaaccctacagtgtaagtttcttttttttctttttcttttttttttttttagacagagtctctctctgtcgcccaggctggagtgcagtggcacgatctcggcttactgcagcttccatctcccgggttcaagcgattctcctgcctcagcctcctgagtggctgggactacaggcgcgccaccacgtcctgctaattttttgtatttttagtagagacagggtttcaccatgttggccaggatggtctcgatctcctgacctcgtggtctgcccgccttggcctcccaaagtgttgggattacaggcgtgagccaccgtgcccggccgcagtgtaggtttctgttactgtcattttccgtctaaggagatggagacagagaggggttaagtaacttgccccggggtgacacagtgtagcccgatttgctgtcaggcagtctgcttccagagtttgcagtcagccatatgtgggtaggatttgtctgagatgcttttgatcatggcaatggagagcttttgcagttatgttgagtttcttagaatcccagctgtttcatttatggctgtgtgactttgcaggttctgtctctgagccttacttacttcatctgtaaaactgggatgattcccacctcacaggcttgctctgagaatgaaacgatcccctgtcagtcaggtgctcagcgtggtgccaaggacagagtcagcccccatggcagtggctctttctgaggatgctgagcagactggcagggcgagcctgggaagggcctggctaaggagctagtcttctgttctgtgtatgaaggatgggccatcagcggggttaagcaggtgagggaggcgaatagctttgcattttaggaggatttcttgggtctcctgcttttgattggacgtggagaggccagaggtaaggtagttgagaggctctgtttagtccaggtgagcagtgatgtggatttgaaccggggctgtggcagccaggatgagaggcagagaagtccaggtagaaatgggaggggcctgataggacttagtgacagatgggaggagagaggggaatgctgattgaaagatctgtctcaggtaactgagagaagggttgtggcattaactgccctagggggatgtaggaggagcgggattgggagtggagccaattcggagttccattgggacatatttagtgcctgtgagtagatctttatctatgtttctttctcttttacttttcctgggtgggtggaagtagaaactagtaaaaatcctgaagcatcaataccgggtgtcttccaggtcctggcaggagaacatctctctgaaagagctccagagccgaggcgtgtgtttgctgaagctgcaggtatccagccagcgcactgggctgtacggacggctgctggtcacctttgagcccaggcgatacgggtccgcggcagctcttcccagtaacagctttacttctggtgtgtgcgtattgacctagacagacattgaaatttactggcattcagaccgtgacttgtaccctcgtaaagactggattaaaatgaccagttggagaataaagcgttgcaatgaagaactcttaatcagaagtccaacaattgatgtgggttttagttgtctagatcagcgttgtccaaaatggtagccattagccatgcgtggtcatcaaatgtttgaagtgtggcttgtaattgccgcagatacagaatacacaccaaatttggaagattatgttaaaaaaacaattgggaatatcttgagtaatttttctgaatactgattacatgttgaaataatatttttcatatattgggttaaataaaaaatatcttattaaaataatttacccatttcttttctttttttttttttttttttgagacagagtttcgctcttattgcccagtctggagtgcagtggcatgatctcggctcactgcaacctccgcctccggggttcaagtgattctcctgcctcagcctcccgagtagctgggattagaggcatgcgccaccgtgcctggctaattttgtatttttagtagagacggggtttctccatgttgttcaggctggtctcgaactcccaacttcaggtgatccgcctgctttggcctcccaaagtgctgggattacaggtgtgagccagtgcacccagcctcttttaaatttcttaatatggctactagaaaacttaaaactacacgtgactgggtgcctgtaatcctagcactttgggaggccgaggtgggtggatcacttgatctcaggagttcgagaccagcctgggcaacgtggcaaaaccccatctctgcaaaaaaatagaaaaattagccagatgtggtggtgtgtgcctgtagttcctgctacttgatgggctgaggcaggaggatcgcttgaacctgggaggttgaggctgcagtgagctgaaatcacgccactgcactctagcttgggggcaaagtgagaccctctctcaaaacagacaaaaacaaaagaaaaactacatgtgactctctttatgtttctatgtaatgggcagccttggtccagctggtaaacatgcaggatgtgtacttctgtattcccaaccctttcttgtgccaagcatgctttggctaaacttgtgccagcctggttggaaaggagggacagttagaagaagcttcttaaccacacgcctgggatgcttgtgttagacaaccacagccttggaagcctggaggaagcagcgttgccagggctgtactttttactatggaagtgtctctgccttgttttggtggtgacggagttttaaatttcatattttgatttcgtagactttagcccaacaattactggaatactggctggatttggagaagatgagatgagggtattgagagaggagagggccaggaggggaacctgtaggttggagtcagacctgagcagagtttgagattttttttttctgagatggagtctcactctgtcgctcaggccggagtgcagtggtgcaatcttggctcactgcagcctcagcctcccgagtagctgggaccacaggtgcccgccaccacgcctggctaattttttgtatttttagtagagacgggctttcactgtgttagccaggatggtctcaatctcctgacctcgtgatctgcccacctctgcctcccaaagtactgggattacaggcgtgagccaccgtgcccggcctaacttactttctttttttttttttaactgttactgattgcatttactaaagaaagtaacaaagataaaatatttgaagtatagtggattttattacagaaaatgatattgaagctttccccagtgtcttgtgtcactgagtctttgtttttgcaggtgatatcgtgggcctgtacgatgctgctaatgagggcagtcagctggccactgggatcttgacccgggtcacccagaagtcggtcacggtggcctttgatgagtcccacgatttccagttgagcttggaccgagagaattcctacagactgttaaaacttgccaatgatgtcacttacaggcgactgaaaaagtaagtggatgggactggaaatcctaagtaccatcttccttcaacacagctaatccattcttacatgcctgtcgcacaaaagagagcaacaagtcatggtgtgggtgtggccctcatgggagttggtggcagcattggggcagaggctcgggcactgaattgcaggtgttctaattttagttttctcccttggcagagccctgattgctctaaagaagtatcattctggcccagcctcctcactcatagaagtgctctttggcagatctgctcccagtcctgccagtgaaatacgtaagaacttctgagttttcttttttggttgaaatcactactgaaagtatagagaacagtgtgaccttgggccgttttaataagagtttgagaaagtatgactagaagaagtctccctgaacatctttgtaggagtgtctcggagggccttgcatgaactggaccgtcctccttgtactcagtcctaatgaactggctcgttccattgaggttttttttttttttttgagacagaatcttgctctgttgcccaggctggagtgcagtggcgcgatctcagctcattgcaaactccgcctcccgggttcaagtgattcttttgcctcaacctcccaagtagctgggactacaggcatgcaccaccacgcctggctaatttctgtacttttagtagagatgaggtttcaccatattgaccaggctggtctcgaactcctgacctcatgatccacccgccttggcctcccaaagtgctgggattacaggcgtgagccaccgtgcgcctggccccattgagtttttagttaaaaaaatttttttttggcggggggggtggagggggggggcggatggagtctcgctctgtcacccagactggagtatagtggcatgatcttggctcactgcaacctccgcctcccgggctcaagtgattctcatgcctcagcctcctgaatagctgggattacaggcacacgccaccacgcctggctaatttttgtatttttagtggagacagggttttgccatcttggccaggctgttctcaaactcctgatctcaagtgatctactcacctcagcctcccaaagtgttgggattacaggcatgagctaccgtgcctagcctccattgagttttaaaataatgttctttttagtgtatataaatcttttattttgaaatgtaatgatatttcttttagcttagtactttgtgaaaatagaggaaagctgatcatactttatgcttgatagcaattttaggttggaaaaatcttttttttttttttttgagacggagacttgctctgtcaccaagagtggggtgcagtggtgcgacctcggctcactgcaacctccgcctcctgggttcaagtgactctcctgcctcagcctcccaagtagttgggattacaggtgcccgccaccatgcccggctaatttttgtattttttagtagagacaacatggtttcaccatgttggtcgggccggtcttgaactcctgacctcaggtgatctacctgcctcggcctcccaaagtgctgggattacaggcgtgagccaccgtgcccggtctgtgcactttttatgtcctttgctgttcttggagaacgtcatttttgggaggagtttgttgtctcatccagtcactgcccatgatttaacagtcctgtgagtattggacttggcccatttgtaaagcggtggctttggccaagcctcatgcaagggttaccccacagctattttctatcatactgtgctaggcaagccagtgttgtgttagcttcaaacacacaggacgactttgtagcactctttcagcatgcagtccctcaagatgtgttgtttttaaatactcattttctttctgcattttcctttggaaattctaaatgcagtcacatttcagtaatgctttatttctatgaaatatttactgaagggctcctatgcgtcacaaactgaattagaaatgtatttactgtgtcttagggactctggaatggcttggctagagcacgggaacgtggggttcaatggcctgttactagctgcacaccctgccgtcttccatgttggcttcagtctcacaagtcagggtcagcaaacaccctttgcttatgagctgctccttatccgataggcgtctctgtgtagtgggcaccatgtagcgaatgccatggctgcagggctgagcacagcaccacctgctctcaagagcacagcataagaagcaggtcttggccaggcgcggtggctcacacctttagtcccagcactttgggaggctgaggccggcagatcatgaggtcaggagatcaagaccatcctggccaacacggtgaatccccgtctctactaacaacacaaaaaattagctgggcatggtggcactcacctgtagtcccagctactctggaggctgaggcaggagaatcgcttgaacccaggaggcggatgttgcagtgagctgagatcatgccattgcactccagcctgggtgacaagcaagactccatctcaaaaaaaaaaaaaaaaaagaagcagatctttagtcttggtcgtggaagattcaaaatagaaacacgatcctagaatttgttaatcattgtttttgtcatttgtctttatcattttatgggcactttaaagagggacattatgttagaaaggaatatgctctaggaaacatttccttcattcctgttactctagagcaggggtttttcctgtaaagggatggattgtgaatgttttgactctgtgggccatatcatacgtaccgtctacagctgcctgctgtagacggtatgtaaatcaatgggtgtggctgtgctccagtaaaactcaccaaaaccggcagctgactggatgtggcccacagtttgccgattcctgttcaaggagaaaccttgtgaaaataaagcagacttaattttgtgttgattgggttgcccctggggaggtccggcgtgttcctgacaggcatcactcatcccccggggcacacactctctgaggaggaacacccacagagctccccagagcacctgagcctcacgctgctgcttcttccacagacccgctgacattcttcaacacctgcctggacacctcccagaaagaagcggttttatttgcgctgtctcagaaagaacttgccatcatccatggacctcctggcactgggaaaaccacgactgtggttgagatcattcttcaagctgtgaaacaaggcttaaaggtgggcagtgcatgccacttccctgtcagagcccgtccgctcctggtctgttgtgttcagtgggtgctcagcgacctttcagcctcagttctggtgggatgggtcaggaacattactgagcatttattgattgagccttggactgcccctgtttgatttccagcatccccgtgagtcttggggagcacgcgcaggagccgtagcctgggttgttgaggaaggctcctggagccctttgaggcgtggcagccttcgtgtcggccaggccctgggtcagggtttatttgttcaccacgcttgctgtgctcgtgctcctccgtgtgcacgcccgtgcaggggcctggagttggcctgggcccggtctgtgcccgtcaggagctcagcgtctggttgaggtgttaacagctaatcagatagtctggttaatggatacgatgcacatgtccagtgcacagatgaatgattttctgagatgtggaatataacattgtacattattattcacttacattttaaattttttttatttttattttttggagacggagcctcactctgtcacccaggctagagtctggagtgcaggggcgggatcttggctccctgcaacctccgcctcccgggtttaagtgattctcctacctcagcctcccgaacagctgggattataggcgtgcgccaccatgcctggctaatttttgtatttttagtagagatggagtttcaccacattggccagtctggtctcactcctgacttcagttgatctgcctgcctcagcctcccaaagtgctacgattacaggcatgagccactgtgcccggcccagttacatttttaatggctgaaaaaggatcttttatcatcatctgcatcagcttgagtcattgtggatcttggcacatggagagtttttttttttttaatttatttatttatttttaattatactttaagttctagagctttttgaaaaaactttctggtttataaaaatgttggataatgatgagaattggaataatggccagttttgtagtttgatgtttactgggtgctgcctaccaccaggcatcgttctgagcacccatttaatcctcactgaaaccctgtgccaaggcaggtggttagtggccccagtttgtagacaaggaagccgggtgtggtggctcacacctgtattctcagtactttgggaggctgaggcgggtgggtcacctgaggtcaggagttcgagaccagcctggctaacatggtgaaaccccgtctctactaaaaatacaaaacaattagccgggtgtggtggcacacacctgcaatcccagctacttgggaggctgaggcaggagaattgcttgaacctgggaggtggaggttgcagtgaactgagatcgcaccattgcactccagcctgggcaataagagcaaaactccatctccaaaaaaaaaaaaaaaaaaaaaaaaaccaaaaaaaccacgaggaaactgagaaacaaggtcaaatgcccagcactcagagtggcagagccagcaatggaactcgggcagagcgcagggaagcctgtcacttgcttaaaagattacgtccagcggttttccaatgtgtgagtgtctttgcacatgtttgaaaactggtaagaaaagatgacttattggaagggagcagattggtggttttaattgggtgccgactattcactaaaagtgagccaagacacacctgtgtgcacgctttggtggagatatgcctttggacatgccagtggggttggaggcgatgctgggcatagagagtgggtggctgagcctccttcctgagctgctgtttgttttgagcttaagggcctgtagagactctgcctcctctgctggttgccacctcctcttttttttttcctcagagatggggtctcactatgttgtccagggtggtcttaaactcctgacctcgcaccttggcctcccaaagtgctgggattacaggcatgagccaccgtacttggccagcgtctcacttttaatagtggagctgtggctgggtgcagcggctcatgtctgttatcctagcgctttgggaggccaaggggggaggatcacttgaacccaggagtttgaggtcagtctgggcaacatagcaagacccagtctctacaaaaaatagaagaaattagctgggcatggtgatgcatgcctgcagtctcagctacttgggaggctgagccaggaagatagcttgagcccagaaagtcacgaaacaccaggagtgtggtctaggtcttgctgctcgccacacagaaagccagttactgaggacaagtattgccaaggaagaggctttataatcagatggtgcagccgaggagatgagagctcatcctcaaacccatctccctgactaaaactaggggtttatataacagggaagaaatgtagcaatgtgtaaaaaacaggaactagggagggcaaggaggcacctgtagtggtctggtgattttcagatctttgatatttttttgagaggcctcaatgtccttttctgaggaagaaactcacataaataagtttgaagctttaacagcagatggtcaatttctctttatccaaaaacaactgtccatgggactattgagccggtttctccatgatcctgccactgcgctccagcctgggccatagagcaatttgtttcaaaaaaaaaaaagaaagaaagaaaaatatagtggagctacagtgttcgggagacaactgtgagtccatttgaaacgggaagggttcccttgtccccctcgcagggcgtgggatgggggagtggctcgcttcttcagtgtcctgctgctcagacctgcaggggagcatacagatgggcaggctgtgggctctgacctcatggcagtgtctaggggtgaatgtttacagctctgtgagttacagggtgctctcttagtttgccgtctataggtggcttatgttaaccaactcaattagatccccttccttgtcacaaggacagagggctttctgtatcctgggttcttgccttggggtaccggaagaatcggatcacacgtgggcttggaaaatgaatgcaagatttgattgagagcacgtgtgtacatgccttgtgcttctttctacctttaaggctttttgttgttttagtgtcaacttcagtggtttgattacaaagtaaatgaccagatcctaacttgcggttcccaggttctgtgctgcgccccctccaacatcgccgtggacaatctggtggagcgcctggctctgtgtaagcagcggattctgcgcctgggacaccctgcccgcctcctggagtccattcagcagcactccctggatgcggttttagcgcggagcgacagtgcccagattgttgcagatatcaggaaggacatcgaccaggtctttgtaggtgtcatggccagtgtccatgtggggcgtgggcttctgatctgcagtctttttaaggagcaaatttatctgtctgcattcctcttgaccttctcttgcttctgtcgattcctttaataattttaaaaattgggctgcccttttttttttttttttttttttttttttttgtgacagagttttgctctcattgctcaggctggagtgcagtggtgcaatctcggctcactgcaacctctgcctcctgggttcaagcgattctcctgcctcagcctccctagtagctgggactgcaggtgcgtgtcaccacgcctggctaatttttctatttttagttgagacagggtttcatcatactggtcaggctggtcttgaattcctgacctcaggtgctctgcccgcctcggcctctcaaagtgctgggattacaggcgtgagccaccatgcctggcctttgtttttgtttttttttttgagatggagtctcgctctgtcacccaggccggagtgtagtgctgctgtcttggctcactgcaggctccgcctcccggggtcaagtgattcttgtgcctcaccctcccaagtagctaggactacaggcatgtgccaccacacccaaccaatttttttatttttagtagagacgggggtttcgccatgtgggccaggctggtctcgaactcctaacctcaagtgatccacccgcctcagcctcccaaagtgctggattacaggtgtgagctactgtacccagccagggctgcccatttttgatagagaactatacttagcatttaacagagtctcaactgtcttcagagcacttggttagtaggtgtgaggaattgtaaagaaaacattttccggccggatacagtggctcacacgtgcaatcccagcactttgggaggccgaggtgggcggaacagttgaggtcaggagttcgagaccagcctggcccacatggtgaaaccccgtctctactaaaaatacaaaaattagctgagcatggtggcgggcacctgtattcccagccacttgggaggctgaggcaggagaatctcttgaacctgggaggtggaggttgcagtgagccatggtcactccactgcactccagcctcggtgacaaagcaagactccgtctcaaaaaaaaaaccaaacattttcctgggtgtctttctgtctggtcctgttgtcttgctagtagtggcaatgggagaaaagtagcctgaacataaacattgaaaaaattggcatcctcctagagctttccgtccattccaccttctccttgtgaaagaggacaccagtcggctggagaagtagggtggtggtaggtttcttccatttgagagggccttttccccccagcccgacactccgacacttttgagctctttggttctcccgtggacgctttgttgtagtgtctgtgctcacgtcctgtcctgtgtgctcactgtcggtactggtactccttggtgtcttagtctcagaggagtgatggcagcagggagggtgctggagccagtctttgctctaatgtctgtgctcacatcttgtcctgtgtgcttacggtcagcacctgcaccctcggtgtcctaacctcggaggagtggcggcagcagagagggtgctgggagccagtctttgctatgggcccgtggcatcgaggctgcttgcgtggctgcttgcatggcagcgtggggatgggccgcctgctgggagggggaggaaggatagatgcatgtcatgcaggttcgaggtggtggaggaatatgcatgatgtttgctcatcacaaatgtgggtgatttcatttcctgttgaaattggtgcttttgaggattttctttctctgtaaattatacacagtaaaatacctcctgtcacccctgctaaaactccatcccccaaagtagaaatcacaaataaggaaaggttacatcttttttttttttttttttttttttttaaagaatcttgctctgtcgcccaggctggagtgcagtggtgcaatctcggctcactgcaacctccgcctcctgggttcaagcaattctcctgcctcagcctcccgagtagctgggattacaggcacacaccatgcccagctaattttttgtatttttagtagagacagcgtttcactgtgttggccaggctggtctcgatttcctgagctcgtgatctgcctgcctcgggctcccaaagtgctgggattacaggcatgagccaccgcgcctgaccagttccatctttttgagatattaaagtaagttgaggttttgattatgggttgaaaaaattttttttaatctcttgaaaaatgtagaataatatgtgaagctcagtctctctattcgtaagctcctacaacccaaatcctggggcgggcaagcgctttttcttccccaagatcaaatgcctgtgtgcagggcagggctggtccaatttctggtctcctctgagttaaaaacgtggtctctttccaggacttaaaatgtctcttcctgtgacttcaaggcaagtgaggatgtaaggtgcttagttgcgtgtgtgtatctgtgtctctttcaaggaagatttctgagtgttttttacggagtttattgaactggactgaatgatagaagcacttcataaatagagttgaagaagtacaaagttggactgaagtaagtgtatctcctttgtttttctttataggtgaaaaacaaaaagacccaggataagagagagaaaagtaattttcgaaatgaaattaagctgttaagaaaagaactgaaggagagggaagaagcagctatgctcgagagcctcacttcggcaaacgtggtccttgcaacaaacacaggtgagggggcgtctccatcctgcctgtgtggcctcgaagaaggagttggggtgtgttccctcttctgttttctggaagagtttgtttagaattggtatttctttcttaaaagttgggtagggttcactagggaaggcctccgtacctgcagttttcttcatggagaggttttgaactacaaattcaacttcttcaatagataaagggctgttcaggttgtttttcttccatgagcttgtagctttcaagaagtttgtccattttatctaaacggttgaatttagaggcatgaagtggttcctaatatgcccttattcttcttttactgtctgcaggatctgtcatgatataacctgtattattcctgatactggtttgtgtgctcttttttttcctgctcttctggctggagggtttttttttttaaatttagattttatatatacatacatatatattttaagagacagggtctctgccaggcgcagtggctcacacctgtaatcccagcattttgggatgccaatgcgggtggatcacctgaggtcaggagtttgagacctgcctagccaacatggtgaaaccccatctctactaaaaatacaaaaattagccaggagttgtggtatgcacctgtaattccagctacttgggaggctgaggtaggagaattgcttgaatcctggaggtggaggttgcagtgaaccaagatcgcgccactgcactccagcctgggtgacagagcaagactccgcctcaaaaaaataaaaaccccaaaaccaaaaaaacaaagcaaaacaaaaaaagagacagggtctcactatgttgcccaggctggtctcaaactcctgggctcaagggatccccctgcctcagcctcctaaagtgctaggattacaggtggacccaccgcgcccggcctggctgcaggtttcaattgatcttcccaaagaaccagcttttgctttttctctattgtttttctgtttcatgtatttccacattgatctttatcgttttctgttttcttgggcttgttctttttcttgcttcttaatgtagaagctgagatcattgatttgagacctttctttttttctgatacgagcatttagctccataaatgtctccctaagtactgcttttgtggcatcccacaaattctgatctgttatatttcattcatttcaaaatgctatctcatttgccttttggttttttagaagtgttatatagtttctagatagttgggaaatgtccagatatctttctgttgttgatctctcctctcctctcccctcccctccccgtccccgtccccgtcctgtgtgggatctcactgtgttgctcctctcctctcctctcctctcttcttctctcctctcctcatccctgtccccatccccatccccgtcgccttccctatccccatccccatatgggatctcactatgttgcccaggctggactcaaactcctgggctcaggtgattctcccagctcagcctcctgaggagctggggctccctgcatgtgccactgcacctggcttactttctgttattggtttctaatttagttccatgtggtcagaggacatactctcatgacttggatagttcatcgagacttactttatggtccagaatctggtctctgtctttgtgagtgttccatgcacacttgatagaacgtgtgttctgattttattgagtgttctgtaagtagtcagatcatgatggttgatgatgtggttcaagtctgctgtattgagagagggacgttgaaatctctgaatataactggacatgtctgtttttcctgcagttctattggtttttgcttcatatctttggaaactcttttactggggtcataaatgtggggctatgttttcttgattaattaacaaatttatcattatgaaatgaccttctttatccctgataatattctttttttgaaatctactttgtctgacattaaaataatcatttcagcatttcagcttgcttttcttttcattatggtcaaaaacctgtaaaatttaccatcgtaaccctttttaagtctgtagtacagtagcattaattgtatgcatgttgttgtacagcagatctctagaacattttcatcttgcagacctgaagctctggatctgttgaacagcaattccctttttccctgtcccagatcctggcagtcaccattctcctgtctgtttctaagaattttattattttacatacctcaagtaagtggaaccatgcaggatttgtcgtcttgtgactggcttgttttatttagtgtaatgccctcatggttcatccatgttgtccagctttctttttatttttaagagacagggtcccaatctgtcacctaggctggagtacagtggtgcggtcaaagctgtactccagccttaaccatgaatgtgggacctccagcaatcctcccacattgacctcctgagttgctgggattaacaggcgtgagccattgcacccagcccccacctttctttcaactagtgttagtgtgatctatctttatcctttttcttagaacctatttgtgtctttatgtttaaagtgatcttttaaaaaatatttgagacacggtctcactgtgttacccctaactggagttgcagtggtgcaatcatggctccctgcagcctcaacctcctgggctgaagtgattctcccacctcagcctcctgagtagctgggacaacagacacgtgctatcacaccctgctcatttttttgatttttaatagagacaagatctcactgtgttgcccaggctggtctggagttcctggactcaagtagtccttccacctctgcctcccacagtgctaggattacaagtgtaagccactgtgcctggcctgtttatttctttttaaagagagagtcttgctctgttgctcagactggagtgcagaggcgtgatcataacttgctgtagccttgcattctttggctcaagtaatcctcccacctcagcttcccgagtagctagtactgtaggcatgagccattacacctggctaattttaaaaatgttcttttcttttttccttttagagatggggtcttgcaatgttgcccaggctggtctcaaactcctggcctcaagtgatcctcccactttggcctaaagtgaatttttttgtagatagcatatagctgggccttgctttattatccagtttgacagtttctgccgtttgttctttgttatattttcctcctcttctgctttcttttaattaattaattgaacatttctttttttttttacataagttctcactatgccccatgattgaagatttcttaggaatttatttttatttatttatttatttttaaaagataatttatagagacaaggtcttgctgttttgcccaggctggtctcgaactcctggactcaggggatcctcccaccttggccaccttggtgctgggattataggcatgaaccatcactcctggcctatttttgtttttgttggcttgttagctatatatctgtatttgattctttcagtggttactttaggccttacagtatgtgtcttcaactcatcatagtctgttcatatgccacctcacatatagcataagggctttatgattgcaagctgacatttctctcctcttagcatttatgcttttgtcatatatttttcttatacgcatattctaagacttacaatgcttgttcttgttttaatggtgaattatctttaaagagatctaaataataagaaaaatgtcttcgtatttgcccgtgtagttaccacttttggcgctttgttttcttttggtttaggtcgtgtatttccacgtggcatcattttccttccacctgaaaggcatccatgatcatttcttgtagtgtgggtcagccggtgatgcagtccagtagcttttgtatgtctgaaaatatcttgtttcacctgcagttttgaaagatatttttgctgcgtgagaattctagattaacacttttccttgcaatattttaaagatgttgtccactgtcatctgcctgagtatttctgaagagaaattggtcatccttatccttgttcctttgtatgtaacgtgtcttttttcttctgtctgcatttaagattttttcagccaggtgtggtggctcatgcctgtaatctcagcactttgggaggcttaaggcgagtggatcacctgaggtcaggagtccgagaccagcctggcccacatgtactaaaaatacaaaaaaattagcagggtgtggtggtgggcacctgtaatcccagatgctcaggaggctgaggcaggagactcgcttgaacccggaggcggagattgcagtgagctgagattgcaccactgtactccaacctggacgacagagccagactccgtctcaaaaaaaaaaagaaagaaaatgatgctttctttatcactggttttaagcaatttgactatgatttgccctggtgctatttgcttcatgtttttttttgagacagagtctcactctgtcacccagactggagtgccgtggcgcaatcttggctcactgcaacctctgcctcctgggttcaagcgattcttctgtctcagcctctggaatagctgggagtacagatgcacaccaccacgcctggctaatttttgtatttttagtagagacggggtttcaccacattggccaggctgctctcgaactcctgacctcgtgatccgcctgccttggcctcccagagtggtgggattacaggtgtgagccaccgcacccggcctcatgttttttaacattgtggtgagaacacaacatgagatctaccctcttaaatgtttaagtgtactatataatattgttaactatagacgtcgtgttgtgcagcagatctatagaatttatcttgcacagctgaacctttatacttgcttattgctgagttcactaatttccttctttcatgcctaatctcacatctgtcccatccagtgtaactttcatctgacatccagaaataagagtctttttatatctttgatgttcttatttaatttttttaacatttggaatacaactataataactgttttgaatattctagactattatttctctatcacctgtgtcatttttggtctctttcgatgaactgttccccttgagtggttgtgttttcctacctttttgtaggcctgctaatgtttgtttggataccggacattgaggattttacgttgttgcatgctgaatattgaatatttttgtattcctataatctttttttttgtttttttgttttgtttttttgagacagcgtcttgctctgtcacccatgctggagtgcagtggtgcgatcttggctcactgcaacctctgcctcctggattcaagtgattcttttgactcagtctcccgagtagctgggactacaggcatgcgctaccatgcccggctaatttttgtatttttagtagaggcgaggtttcaccatgttggccaggctggtctcaaactcctgacctcaagtgatctgcctgcctcggcctcccagagtgctgggattggtgtgagccaccacgcccagtctcctataatattcttgtgcttggttttgggaagcagtaaagttacttgtaaacagtttaatccttttgagtcttgcttttgagatgtgttagggtctggatcagtgcttagtccagggttaatattcccctgtactgaggtaagaccctttgagctttctacccaatgcaccctgtatcatgagatttttgagtttgactggtgggattaggtgctgttcccggccttgtgtgagcactgggcacttttctctcattgttttggatggtatttttcccaggcctggagtagttttctcctaagcatgtgctaatcacaatacccaagggaatctcgcagcagatctctggggttttttctctctgtgtagcagtctctttggcattttgtcctggaaactctgcctgcattggtcgccacagattctcagccttgtctcctcaactcagggagccttctaggttctacctggctgccccttctctgtactctctcctggaaactctcaaggcagaaagctggggtaaccccagggctcacctcctttgtctctcgttattcaggggttacttttctttgttacctgttgtctagtgtcctgaagagtgtggtttcagttattttaaaaaaattttaaataactggctgggggggttgttatttgagatgggagagtaaatgcagtccctgctattccatcttgggcagaggtggaggacgcacattctctcttgtagccaggagagagcactcccgttacagagctgtgtaccttgtgactgttgtgggaatcttgtcaaccactcttttggggtttaggaatgtgtttgtgagacctctggtttgttcctgtgtgataatagtaccacaaaggtagcagctgaaaacaacccacgtttattattctacagtttatctgggcggagagcccaggcccggctgaacaaggtgctgtgctcaggatctcacaaggctgcgatcgaggtgtcagtgggggctgcagtctcatctgaaacttgcctggaaaggatctttcttcaggctcacgtggttgttggcagcattcagttccctgtgggttcttgctgggtgttggctggaggccgctcttagctgtttgcccttaggccttgccaacatggctgcttgcttcctaaaagccagcaagagacatagtctcctagcacaatgggttatcgtcttatgtaatgttatcaagggtgtcacatctcatcacctttcccctgttctgtggattagaagcaagtctcaggccccacccacatttgaggggaagggattatgcaagatgtgaataccagtggccaggatcctggggcataccttaaagcaagtttgtctaactaaaggcctgcaggccacatgtagcccaggatactttgaatgtggcccaacacaaatttataaactttcttaaaacattatgagatttttgtctctgtgttttttttttaagctcatcagctattgtcaatgttagcatattttatgtgtggcccaagacaattcttcttcttttaatatggccctgggaagccaaaagattgcacatccctgccttagagtctgtttgccacaacctctcagctggtgggctgctctaaggaacagagtccaggttctttttttttttttttttttttgagacagcatctcactctgttgcccaggctgtagtgcagtggcacaatcttgattcactgcagcctcctcctcccgggttcaagtgattcttgtgcctcagcctcctgagtagctgggattgcaggcgcatgccaccaggcccagctaattttttttgtatattttgtagagactggatttcaccatgttggccaggctggtctcgaacttctgacctcaaatgatgtgcccaccttggcctcccaaagtgctaggattacaggagtgagccaccacacctggctagattctaattggctttgtagttattgctttatccagttgtgttttaaatcagacaggagaaaaaaagggtcacaagcagaaaatacatttatggggtcttttatatctacccatgtagttgcctttatttcttcatgtagatttgagttactctctccaaaggactccctttggtatttcttgaagggcaggtttgctgtgacagattctctaagctcatctttatctgggcatgtcttagtttctccttcattttttgaaggatagtcttgctgggtatagaattcttggttgacagtctttcagccctttggacgtgtcattccattgcctgcatactttctgatgggaaagcagctattgatctcattgagggctccttatatgtgacaagttgctactctattgctgctttcaagtgtcgtcttttgtcttttgagtgtgtgattatcatgtgttaggtgtggatctctgtgagtaaattacatggagtttgtggaccttcttggatgtttagattcgtgtttttcatcaaatttggggagttttcagccattattttttcaggtattctttctgcccttattctactctcgtctccatctacaactcccattatgaatcagttgattcacttgataatgccccacagatctctgaagctctgttcattttcctccatttttttctttctgatcttcaggctggatcttctcaatggacctctcttcaagtttgctgatcattctctcctgttgaatccttctagtgaatttcagttattgtacttttcaactccagaatttctgtgtggtgcttttttttttttttttttttttttagatggagtctcgctctatcgccagactggagtgcagtggtgtgatctcggcccaccgcgacctctgcctcccaggttcaagcaattctcctgcctcagcctcccgagtagctgggtctacaggcgtctgccaccacgcccagctaatttttgtatttttagtagagactgggttttaccatgttagccaggatggtctctatctcttgacctcatgatctgcctgcctcagcctcccaaagtgctgggattacaggcatgaaccaccatgccagcctctgtgtggtactttttaaaaaattattcctgtctctttattgatattctcttatttggtgagacattattctcatattttatttctttagacttgtttcccttttgctgtttgaacatacttagaatcgctgatatttcaagtctttgtctagtaagatcagcatctgggctttctctagtatccagtttatttatttatttatttaattttttggagatggagtctcactctgttgctcaggctggagtgccgtagcgtgatctcggcctgctgcaacctccgtctcctaggttcaagcgattctcctgccttagcctcctgagtagctgggattacaggcgtccaccaccacgcctggctgatttttgtatttttagtagagatggggtcaccatgttggccaggcaggtgtcgaactcctgactcaggtgatctaccaaccttggcctcccaaagtgttgggattacaggcgtaagccactgctcctggcctcaagtatctgttgattgccttttttcccctgaatattggccatattttcctgactctctacgtgccttgtagttttttgttgaaaatggatgattttaaataatagaatgtagcaactctggaagtcaggttctcttcccaccccggggtttgttgttgttgccacttgttgtttgtttagtgacttttctggactaattctgtaaaatctgtattttttgtcttgcgtggccactaaagtctaatacaacttagtggccagctagtgattagacagaaatccccctgaactcctggagccataaatgccccagtctttgctgagtggctctgtgtgtgtgcttgggcacacctccaacacagagccaggctgtttgtgactctcagctttcacttcccgtttgtgcagtgcgttgagctgagccacagatgagagctgagggcctcccgaggtctttcctgagccctgtacatgtgtgtggccttctagattcccaggaatatgttggagcttttcaaagccccctgaggatatctcattccccagcttaaaactttggttatcctatcatttgctccgaccattattctctgtcttagaaaactgtgatgttcaacaattacctctgattgtttttgacagacaccaccagggaaaaggctgtttgtattacatgagctcagataaagtcttttgagtgtggtctaccagaggattcccagacagatgagataagcacagtttctggagtggggcttttcaggaatgccaaccctgttctgcaccccccggggctggcaggtgctggttttcaccctgacttcaggctgagggtttcaaggctgccatagggatggctgggaatagggcaggtttcagtgccaccgagctcccgtgctgacggggattcagatgcttttctggaatgaacacttccctgattgctgcagcctttggcgagtttccagagttctgagaaagttgattctgaccatttgccgatgtcttgttgctttcgtggaggagagaattctcagagcttgtgagagtctctttgctgacatccttctctggcttcaaggacagcacttgctggaaatcaggaggcatcccctgagccccacaaggtcagcttgtggcctttgtggcctttctgttccctgctggttgttggggaactagagtgagttcctggaagtgcttctccatcatgaagtctggtttctctctgtactgtgaagtcacatctgtgacctaaacatgtctcttaattatccaaagcccagccaagttctgtctcctctgtgggtgctttttctagccatctctgaagagtcaatcttgagaaatttctcagtatgtcttcatcatggcattatgtacatatcattagcataattcactcagatggaaggcagaaagggtgggaactaacattgatcacatttatggaaacctgccttctcgtgctgggcactttatatatgttatctccctttgtggtgcaatctcatgacatgcagtcattgtccatgtttgtgggtgaggaaacaggcttaggggtgggaggctcgcctgaggccccacactgttggctggagacagcgtggggcctgagtcttgctcacagcctgaacgctgcactctgctgctccgcgtcccaggaaggaaaagctgctgcagtgggtttgtgttgccaaatacatggaggcttttttctgggtgagtgcccagcagttgattgttctgtatgtccaagaaagtcaccttagaaaagaattaggttctaaatttagaaaataattgagttacaatatttagaactcagacagcatcccttgggcttggggtcacagccccgaaggggtgattaattttgccaattgaagatcagaggggcctgtggccagagtacccactgcttaagacacaggtgagctggaaccatgagttttaaaatgtcttggccaagtttttattacaagatacaaatttatcccacacagttgcaatgcaagccttgatgaaaccccagcttgatggtgtctcctctggtgtggctgcgctgttgggaagcagctgtgcccctggagactcccggctccctgtttccaccaggtgcgtctgccgatggccccctgaagttgctgcccgagagctacttcgacgtggtggtcattgacgagtgtgcccaggccctcgaggcgagctgctggatccccctgctgaaggccagaaagtgcatcctggcgggcgatcacaagcagctgccccccaccacagtctctcacaagtaagacccctttgcctcacatgcccttctctgcccccgccctcctgcggtccttccacgctcagccaggagccccaggctcaccaggagcccctgcggtgcctcctcggtttctcggcacagctcctaccccgtcttacagcattcattcatctcaacgtcgcatctgtgagatcatgtactccccgagggtggggtcgcatcaccttcatctttgtagcttcagaactggacacagggcctggcacgtttctggtgttccgtccctgcttgttgagttgaatatcccctcatgaaccaggcaggtttgatggtagagtgccgtgagagccttgggctgggctaactggcctctgggtggatgtgggttctttagtcttcaatctgtagacacatccctggatggagggcactgggcagagactcagcaaaccatggttctgccgtgcggagacaccggagccctgatgcagtggcctggcccaggctgtcctggtggagacactggagtcctgatgcggtggcctggcccaggctgtcctggtggagacactggagccctgatgcagtggcctggcccaggctgtcttggtggagaaagtgctgcccgcccccccagcccccctctcacctctccccatactacccagcactcactgcttcagccacacaggcctcgctggcccctctgcctggaccactcctgcctgggtgtggcgggcgtgccctctctccagatgaaccctctgcatggtatcccttgactgcccttacacgaggcctgtgaacagggcctggcacgtgggctgtccttggacaagtccctgttcagaaagttgtccctgaggaatgaaacacttctgagactcctgcatcctggccctcacttccaccgggggaagattgatttcttcagatttcggttgccttttgaaacacttctctgtttcactgtgggtgtgtagaagctatgaactcggaactttcgttgttttccgaggaccggaagaagatgtgcaagaaatcctgtcgagtagaatctcatcacaggggcgtgggcagcccaggagttggcgggtatgtagagagaggtgtcaaaaatagtaattgagagtgagctttgggaggaagatgttggaaataaagatggtggaagaaagaggagtgctgaggaaaggtgctctgaaagatcgtcctggtgggagacacatggggatctagagtggaaatgttacgggatctctggggtgttgatttttctggctggaaacctctgcggccgcagcacctttgcctcagttcttgtcctgcatccaggaagaatgaggtacgcagattagtgaagggtgaagaacagttttatttagtgttagaacagctcagagtagtgggtagctcctctctgtaggcaggtcatccgtcgagtgttcagctgtcagcagaggaggccctggagagggtggctcctctctgcaggcaagtcacttggacatctctgcaggtctctgaagctctcagcagagagcatagatcctctctgccctctccgtcctctggctgtcctctcgccttctctgactgagcccagggcttttatggacctcaagagggaaggaaggatgtgccaattggtccatgggtggccatgggcgggcctggaataggtaccatgagtcctcactctggtccacaggactggcagcccagctgccagccttcaggcccttcctggccttccactgtccccttccactcaggactctgtctgcctcccgctgccattcatggccctggggcttggctccaatcccactctgagatccgagtgggtgctgggagctgagagaggccaggcagtggaagcagacacccatgaccctgcagggatggtgtgggggttgtccttcctggggcgcctgagggtgcaggctgcagagtgggagggcagctacagctgcacccaagagctcccgacccaccacctcagaagaggccaggctcctgcttgttcccggctcctgcctgcttcgtggagcaggaagcccaggtctgcagccacagatctggcggctgcagctgcacgcggaaaggcagatcctacctgttcccggctcccctaagagcaccgggagacgcagatccacagctgcagtttgggcggctgtagccccgcccttctggagcaggaggcctgggtctgtagctgtgaccctgtgtctgggctgcagcggccactctacctcagaaggggcggggctcccactggcttcatggagtgtgcagccacagccagggttccctgctgcagccggcatgatggcagcagccactgccatcagaacctccctgggagcagggaggacaggcgtgggaggacgcttattcagtggcttcttccagggagaggttctcgagatgtccttaaaggaggtcttcatcctctggaaagcctttgggtggaggaagtttggaactgtgcccttgagcagggaccctgttgtgtaggagggagtaggatggtttcgggggaagacgttgggtggcgttccctgcagagagtaggatggttttgggggaagacgttgggtggcgttccctggagagagtaggatggttttgggggaagacgttgggtggtgttccctggagagagtaggatggtttcggggaaaacattgggtggcattccctggagacagtaggatggtttcggggaaaacattgggtggcgttccctggagagagtaggatggtttcgggggaagacgttgggtggcattccctggagagagtagaatggtttcgggggaagacgttgggtggcgttccctgcagagagtaggatggtttttgggggaggacgttgggtgacgttgcgctgagtgttttgggtttgcagttggtactgatcatcgcgtcctgtcctcattttgttggaattaaaaatacgttaatcacagaggtctggtaagtttccagagggaaggtctctgaagtggagctgggtactgccccagggttttggtggggttggtgagccgtagtggtgcagagggtgggctctggcaccaggtggcccgggcaggtcctggctctgcagctcacccgggcagctgtgggctggcagcctcctctgtctcacccttcttacatgtaatatgggagtcataacagtagccacctcatgaggttgttataaggatgaagggagttaatccacatctggcacacccagcagtgcctggcgcctgggcagtgctcagccgctgcagccgttcttggtgggagtggacttgactgctcacagttggagcatgttggagtgtgttcgttccctgttatggctgtgacaaatgaccacaagcttagtggctgacaacaacagattttctgacctccagaaacgacaatctgggggtcagatgtccaaaatcagactcaccggcttaaaatcaaggtgtgggtggggttgcttcttctggaggatccaggggaaaacccatctcctggtcttttccggctcctagaggccactgcttgacttgttgccctttcctctgtctatgaagccagccgtgttacgtcttcaaatctctcccctcggtgaccttctctgactgaccctcgcatgcccccttataagacccttgggttatcacctgggtcatccagggtcgcctccccatcccaggatccttaacttcatctcagctgcagcgtccctgttgtcctgtaagggaacattcacgggccttggcgattggatgtaggtgcctttggggctgtgattcagccaaacccgccccttggttacttctgggtcagaatccaggggagagttgggtcctgaggtttggggcctgtcctcctcacttgctgtggttcacacctggactctggggctcaggcctgccttccccctttctccctcctgggcgcagggctgcgctggcaggactgtcactcagcctgatggaacgcctggctgaggagtacggcgcgagggtggtgcggacactgacggtgcagtaccgcatgcaccaggctatcatgcgctgggcctcagacaccatgtaccttgggcagctcacagcccactcttccgtggcaaggcacctcctgaggtgagtagctcggcaccacccgccgccccatccttctgccctggctaatcctctgcgtcacctgtttctacgcagcaagctaaagtgaagctttctgcatgaaagtcgactctgaggctagctttttggaagagagggtggccttgccctggagagctgggtcaggcccgctccctcccttctgatgtcctgatgtgctcattgtcctcccctacctaagccttttcctcgtgggaggggtcggttctgttgggtggggcctcagtgctgcactgtggccccctgatgtgctccctctctgcctgtgtgccagggacctcccaggtgtggctgccacagaagagacgggtgtgcccctgctcttggtggacaccgccggctgcgggctgtttgagctggaggaggaggacgaacagtcgaaagggaaccctggtgagcttgcttgcagatggccagcttttttgtttaaacatacctccagctctttaaaaataaaaggcactttaattgcctaattccgggaggagctgagagcagttgttgatggccgtgaaaaaacggagccccacactgcaagaggctgagcagcatcgttttctccagggtgagcagaccgctcctgggtgagatgggcctggcagcattttacggccttctcctgaatggtggcccagctctgcctggctggtctggtgatgggaactgtcattgctcagctgacaccaccatctccccagttccacgttgaacaggatgcacggccataggagtcaaaaggccttcattgctgatgtgaaattcaagttagctgggcatggctggtcctggggccctgggacacagatgattagctcccaggaagccacctgagtggaggatcagctggccatgatagacagaaacgtgcccgaaacacgtgttggtggtggatgcccacttggggcgaccttgtgctgctcacccgttctttctttccctccaggcgaagtccgcctcgtcagtttgcacatccaggctctggtggacgctggtgttccagcccgtgacattgctgtggtctcgccatacaacctccaggtacgagggtttccttttgtccctctacagagcagctggggctcacacaacctagagggtgaaagaaaaagggtgatttgttggctgtggtgaccgaaaagttcaggggtagggctgaccttattgtgactggatccagggcccaaatgatgtcgtcaggggcccctctgcgtgttcgtctgcccgccccaccttgccccgccctgcccggtgccccagccttctgcactggcttgtgttctggccagatttgctgggtgaccccggcagcctcaggctgacacagtccttccacgggcagccctggcggaacagcctgtccttttctggcatttcccacaggagtctcagccttgagccttgctggtctggctcaggtcgtgttcccatgcgtatctcatttggtaggaaaaatgtaccttctggagtatcgggaggatctgtggaagtggccatcataccttcctggtgtgcaggcatcacacatagggctgtctgaggctggcggctgtcataagccaaaagaaatgatcaaatgccagggacagggtgtggtattggcgtgcatgcgtgcccgtgaggagggcgaacgggaagcctttccccatggggctcctgcaggctccgcttcccaggtcctggctgtttcacagcgtgaggccctctgctgagctgaagggcagggtcttgctgcgctggcatgggtgggtgaggaaaccacagcccggctggtgtttcaggtggacctgctcagacagagccttgtgcacaggcaccctgagcttgaaatcaagtctgtcgatggcttccaaggccgagagaaggaggccgtgatactgtccttcgtcagatccaacaggaaaggtacggagccctcgccagagtcctttggggacagcacagaagtgaatttattgattgaggggcatattgggtgtctgctgggtgccacactggtttctggcagtgtgctcatggtgcaccttctgtccttagtaagttcacgtcaggcaaagcacaggcaagagagtctgcagaggctcagaggcgtgagtgtgctgtcagggagcaggtggctggggtggcagctgggggcgcagggtgggctgggaatgatcaggacatcctgccgaggaggtgaagccccctgcagcccagagctggcctttagcctccatgcacggagccgcggggtgtggtggtcgtggctgtggtttctgtgctgctgtggtgtgtgggctttcccatttgggatgagcattgttcccagaagcaggtttagtgaacgggctttgtgagacattctcagaaggcagaaagcaggtgaggcctggctagaagctatgggggaaagaagggaaggtcctggaaggtttcttgtgctttctccctggtgccagctgcagccacaccccaggcggcccctgaggctgggcttagaaggaaaggagcgacagaacccagtcgggttccccagagccccactcagtcagccgtggcagccagatgccagggcaggtgtggcatcacggtgccacgcgtggccgggcacccgtgtggatggtgggccacgcgcaggggactacacttttggtggtggttactgataagggcgtaggagcctgactgtgtttcttagtctgaaacctgcttctcactcccctctggccttttgtaggtgaagttggttttcttgctgaggaccggaggatcaacgtggctgtcacccgtgcccgacgccacgtggcggtcatctgtgactcccgtactgtcaacaaccatgcatttttgaagaccctggtggagtatttcacacagcatggggaagtacgcacggcctttgagtatcttgacgatattgtcccagaaaactattcccatgagaactcccagggttccagccacgctgccaccaagccccagggacctgctacgtccaccaggaccggaagccagcggcaggagggaggccaggaggctgcagcacctgccagacagggccggaagaagccggctgggaagtctctggcctctgaagctccatctcagcccagcctcaacggaggcagcccagagggagtggagagccaagatggcgtggaccacttccgggccatgatagtggagttcatggccagcaagaagatgcagttggagtttcctccttccctcaattcccacgacaggctgcgggtccaccaaatagccgaggagcacgggctgaggcacgacagttccggggaagggaagaggaggttcatcactgtgagcaagagggccccgcgaccccgagcagccctgggacccccagcagggaccggtggcccagcccctctccagccagtgccccctacccctgcgcagacagagcagcctcccagggagcagcgtggcccagaccagcctgatctgaggacgctgcacctggagagactgcagagggtcaggagcgcgcaggggcagcccgccagcaaggagcagcaggcctcagggcagcagaaacttccagaaaagaaaaagaaaaaagccaaaggtaagtcaactaataagaacttggggcagtgtcccctcactggggtgctggccccacttggagcccacctgccaccatcaacagaaggaagggcttcctcctggtggcaccgtgcccgtgtcattttagctttattttcagtcatgccactcccagctccgtctgggctcccctaggtcggtgatcaggtcacgggcaccttcttttcccagaccctgtgtgggactcggcaagtaggcagcactcagagcatgtctgcagcagcaggaggggcctggaagagcaaacatggatggctccctggagcccctcgggaatggagcgatcacggggtcccatgcaacatgtggccctggagggtggaggtcttgcacagcgaaagggtgcactctgcctggaagttgtcctgggcaaaatgctcagaaccacaggggctggctgagcgcaaaaccattcttgcccttgggccatgtggagtccctgggtgggctctgccccgtttgccacatcctgcattgtgcccaccccgcaggagctggctaggcgtgcagtgggagttggccaggcatacgctggcagagcgtgtagcggtcttggggaactcctgctactgccattttcctcagcaagagcatgcaggagttccagcttgcattgattcattgaattcctgaagcgaagctacaccagcacatgggtggctttgaaaggggcctcccagagcctggggctccagaggaattcactccccaaacctttgttgagcagctactgtatgccacgctttgtgctagaggacaggactgatgccacccctgcccccaggcactgacagggtagcagagggaagacacatgagctgctttttaattttttttagaattaatagagacggggtctcactctgtcactatgttgcccaggctagttttgaactcctggccgcaagcagtcctcctacctcagcctcccaaaatgctgggattacaggtgtgagccaccgtgcttagccatgaattgattttaaaaccttaaatgaggggccaggtgttgtctttccgtttgcctgagtgacgcgggtcttctccaggacatccggccacagatctgcccacggaggaggactttgaggccctggtttctgccgccgttaaggctgataacacctgcggctttgccaagtgcacagccggcgtcacaaccctgggccagttctgccagctctgcagccgccgctactgcctcagccaccacctgcccgaggtatgtcggcctcccctcctgcgatcaaacagtggggaggggtggtgggttccgtctccaggaagcagaccctgggcagacttgttccagtcggaacagttagctccttacaatctccagataccttcagaaacagaaatcagacactgactcatgatcggttttcagcacatcagggcacaagcaaacaaaatgccttatttgctgagctgcagccacgttagtgggaattctgggcagagggaattttgtcccagtgtccttgggttctgtcagtggcctaatctggtctgtttggcataggggaggtgggaggcagagcctccagcccgtcatctgagctggccatggcccggctgctgtgtcattcattccttgagccggggttgatgagaaccccctgggccaggtgtgggcgcctgtggaaagccgaggtctgaaccagccttgtctctaccctggaggatgctgggcctggtgggggacctgggtaagagaccagcatacagtaccaggtgttgagtgctctgaggaagaaaacccagggccagggtggcagcagggtcacccacccagatgcagcagtcagggtgggcttcctggaggaagtaacactggagctgagacctgtgggacagaaggtgggagctgggctgggaaggacagggccgatggcgggtgggcaggcaggctatccacccttagaacatgcataaagccccaggcccagtcagagagttgggagtagagtgtgaggcagatggctagagttgaggctgggcatgtcctggcgagctgcctggaggagtcaggcctgatctgaaggcagtggggtaccctctagggtgtcagcagggctgtgatgtcaccagtcttggctgcagtgtggattaggtaggcggaggctgggaggcagtgggaagtggccgcagggaccgtgtggtcagtcagggtggtgccatttcctgtgtgcacactcgggggctcccgccacaccagggtgcagaccaccccgccgctctcccgccaggcagcctgcagggtgaaggactgacatggcgggaggagtggccttctctgttggggcgcctgtggccccccagctctttgataggcagagctgcaggatctgcctccttgcccctgtgagcccagcagtgattcttgtgtcctccccagatccatggctgcggtgagagggctcgcgcccatgcccggcagagaatcagccgggaaggggtcctctatgccggcagcgggaccaagaacggatccctggacccagccaagagggcccagctgcagaggaggctggataagaagctgagtgagctcagcaaccagaggaccagccggaggaaggagagggggacgtgaccggccgcatccttgcacgccccgcggagctctctccatggtagcccagggcgctggcagaccatgctccgcctccaccagggccacagaggagcggaggggcctatgggggaggagcggagggccctgttggggaaggttgggtttttggaccccagggataagcttttccgatgtcacaatgtggaggaaagcacctgggggacaacagtgctcgtgcaggtggggcttgggaaatgcacgtcccttccccttactccccgccaaaacccacatcccagcctctggatcctggggaaggttccagtccctggagaatacccagggcctcaaacttgaagtcactcctccaatgtctgggacttgccagctcagcccgttaggatgagggtgctgagaggaaacaggaaacaagactgcgaatggcgctcaggcagggagcagggagtggcgtttggcttgcacgttcccatgtggccagatgctggggccactttccttctgtctgctggtgactgcagtgttccccctcctcctcaccacggggctcctgtgagtctggggggcacctctttctggcctgtgcacctctctctggcttataaaggtgcctggcctgtgccagcccctccttgttgcgcctcaccgtggggaccaggtgagccggctctcccacgtggttgtcccgggaaagctgccccacagcctcagcatcttcagcacttaccgatccagagcctcccggccttctccggtgtcctgtaccaactcttctatttaagagaacctcagatgatgtacctgagcctcagggttttgtttcagagggatataaattatttaaaaattaaatgaaaacgttgcacactgccccagtgtgtctgctgcgggtctaaggggcgcgccctttgcctgttctgctcagctgctgtgccctccccgccgcgtctctcaagccccctctccttctgacctctctgcggcagactctcccggtgcaggctcctctctccttaaatcccattcatgtccttttatgtccccgatggttctgagtgaggttgagcttccctgacctgcacctagggtggctgtcccccagaggccatttacgcagcgcattcggctaatgtgtcctcaggccaggcctgcacgctttgactgccgtgttcttgctggtcctgggcgactctcccttcctacccggactgacgccccaccctgggccactagagcccctcctcctccccctgtcctgtccttgtctgttcccgtcaagtggcttttggactgaattgtttgggaaggggaccaggactctactgcattttgtgtcaagtgcggcagagttaatctggttgcttccataagtgctgagcaccatgcacagaaggctgcagttctcatgctctttgaggacccgtggctgtgggaggcatttgtgtaatgatgctcttgggggatgagggcaccattggaagcatctcccggttgacacagcacacagggacaggtgttcttcctgcaggggcgggggcgggggtgcctgcttaagctccccaacttcccagaatccaaacagctgataagagtcctgccctggggttgacacggatgctgaaagatgttggctcttgggtgggagacagccgacagccacactccctgcctcctaggtagcctgtctgcaggagagaaggaggccaaagagaaacagacccaaaaatgggggtgggagagtgacctgatgacactgtttgagcacctggatgtagccatgcctgacctccaccccattcttcctggtgtcctgagctagtaaatccccttttttagctgaagaagtctgccttgggtttttgtcacttgctgccaaagaatctttgctgacccagtcatgcccacatctgtttatcttattgggcatacccctcccagcatggaggcagtatggaattagtgtcgccccactcattagagaaggcacgattggaatccaagccctgtacctttatccaggtgttcttctgtgggccgtcccaccaggaggagcttgaagtagaggaaggcagccagcatcagcggcgggccgggcgggtggggggtggaagcttctggaatcggagggacctgagtctgaacgccagccagcaccaccgtccactgaggtgtggcctcagcaagtcacctctcctctctgagcctgtttgtcatctatgaagcaggacgggaacagttgctgccgcgccagctgtgctaagtggaagacatgaagtgcagcatcatgtccttgcgggacctggctccgggtttgtcgtctccctggctgtgccccccagcctcctgcatggagccttctccccttcagcactgaccaccgcggcccttggtgatgcattgttaaaatgccagaacccaaggttgcaacataaaaagtttccatttctcatcccagagaggccttgttaggaaaatatttgtagctggttgtttgaaaagtaccctacagtttggcctggatagggtaacccaagaaaaggaaaaaaatttcccaagccaaacgtctccctggccagctggagtgtggtcttgctgacagtagctgcaggctgtagcccacccgggccggaaccgggtggaagagagtagaggactcccttctaaggagaactgcccacctccacccgtgcggctcccctgaggtagccctgccatgtgctcctttccgggggtgccgggcagggcagtgggcacaggcaggagcccctgccagctctgggctttgctcctgagtttacttgggatgtgctgcgtgccttggggcctcagtttcctcctctgtaacatggggaagtaacaagacctgtctc

SEQ ID NO: 7 - DNA, 19 bp, Homo sapiens
  source 1..19: mol_type = genomic DNA
cacttccacagggggaaga

SEQ ID NO: 8 - RNA, 19 nt, synthetic construct
  misc_feature 1..19: note = antisense oligonucleotide
  source 1..19: mol_type = other RNA
tcttccccctgtggaagtg

SEQ ID NO: 9 - RNA, 11 nt, synthetic construct
  misc_feature 1..11: note = antisense oligonucleotide
  source 1..11: mol_type = other RNA
ccccctgtgga

SEQ ID NO: 10 - RNA, 13 nt, synthetic construct
  misc_feature 1..13: note = antisense oligonucleotide
  source 1..13: mol_type = other RNA
ccccctgtggaag

SEQ ID NO: 11 - RNA, 13 nt, synthetic construct
  misc_feature 1..13: note = antisense oligonucleotide
  source 1..13: mol_type = other RNA
tccccctgtggaa

SEQ ID NO: 12 - RNA, 13 nt, synthetic construct
  misc_feature 1..13: note = antisense oligonucleotide
  source 1..13: mol_type = other RNA
ttccccctgtgga

SEQ ID NO: 13 - RNA, 35 nt, synthetic construct
  misc_feature 1..35: note = antisense oligonucleotide
  source 1..35: mol_type = other RNA
gaagaaatcaatcttccccctgtggaagtgagggc

SEQ ID NO: 14 - RNA, 35 nt, synthetic construct
  misc_feature 1..35: note = antisense oligonucleotide
  source 1..35: mol_type = other RNA
gaaatcaatcttccccctgtggaagtgagggccag

SEQ ID NO: 15 - RNA, 38 nt, synthetic construct
  misc_feature 1..38: note = antisense oligonucleotide
  source 1..38: mol_type = other RNA
gaagaaatcaatcttccccctgtggaagtgagggccag

(19) United States
(12) Patent Application Publication
(10) Pub. No.: US 2024/0376478 A1
(43) Pub. Date: Nov. 14, 2024

(54) Antisense Oligonucleotide (ASO) Gene Inhibition and Treatment

(71) Applicant: Vanda Pharmaceuticals Inc., Washington, DC (US)

(72) Inventors: Bartlomiej Przychodzen, Washington, DC (US); Sandra Smieszek, Washington, DC (US)

(21) Appl. No.: 18/578,371

(22) PCT Filed: Jul. 13, 2022
(86) PCT No.: PCT/US2022/073668

§ 371 Date: Jan. 11, 2024

Related U.S. Application Data
(60) Provisional application No. 63/222,336, filed on Jul. 15, 2021.
(60) Provisional application No. 63/224,363, filed on Jul. 21, 2021.

(51) Int. Cl. C12N 15/113

(52) U.S. Cl. CPC C12N 15/1137; C12N 15/113; C12Y 207/10002; C12N 2310/11; C12N 2320/30

(57) ABSTRACT

Embodiments of the invention related generally to antisense oligonucleotides (ASOs) and, more particularly, to compositions and methods for regulating protein synthesis using ASOs. In one embodiment, the invention provides a method of treating a patient diagnosed with myelodysplastic syndrome (MDS) comprising: administering to the patient an amount of an antisense oligonucleotide (ASO) targeted to a nucleic acid molecule encoding JAK2 (ASO-T-JAK2) compound effective to treat such disease.

### CROSS-REFERENCE TO RELATED APPLICATIONS

This application claims priority to co-pending U.S. Provisional Patent Application Ser. No. 63/222,336, filed 15 Jul. 2021, and U.S. Provisional Patent Application Ser. No. 63/224,362, filed 21 Jul. 2021, each of which is hereby incorporated herein as though fully set forth.

### SEQUENCE LISTING

The sequence listing contained in the electronic file titled “_VAND-0224-PCT.xml” created 19 Jun. 2022 and comprising 63 kb, is hereby incorporated herein.

### BACKGROUND

The present invention relates to certain novel antisense oligonucleotides (ASOs) and, more particularly, to pharmaceutical compositions thereof and to methods for treating certain diseases or conditions in which regulating protein synthesis using ASOs can have therapeutic value. More particularly aspects of the present invention relate to the treatment of known disorders such as polycythemia vera (PV) and myelodysplastic syndrome (MDS), as well as Charcot-Marie-Tooth type 2 (CMT2) disease.

PV is a myeloproliferative disorder characterized by erythroid hyperplasia, myeloid leukocytosis, thrombocytosis, and splenomegaly. Most individuals diagnosed with PV carry a valine-to-phenylalanine mutation at position 617 (V617F) of the JAK2 gene.

MDS is a group of clonal hematologic stem cell disorders characterized by ineffective hematopoiesis, often resulting in anemia. Some cases of MDS have been associated with the same V617F mutation of the JAK2 gene and specifically with an increased platelet count.

The JAK2 gene codes for a non-receptor tyrosine kinase involved in cell growth, development, differentiation, and histone modifications, as well as cytokine and growth factor signaling.

Charcot-Marie-Tooth disease (CMT), also known as hereditary motor and sensory neuropathy, causes damage to the peripheral nerves and nerves involved in muscle control. Symptoms are progressive, often beginning in the feet and lower legs and then the fingers, hands, and arms.

Nearly all cases of CMT are inherited. More than 40 genes have been associated with CMT. Some genes are associated with more than one form of CMT and some forms of CMT are associated with more than one gene. As such, it is possible for an individual to suffer from more than one form of CMT as a result of more than one inherited mutation. More than half of all CMT cases are caused by a duplication of the PNP22 gene on chromosome 17. Other forms of CMT are caused by X-linked mutations.

CMT type 2 (CMT2) is a less common subtype of CMT affecting nerve axons. Most forms of CMT2 are inherited in an autosomal dominant pattern, though some forms, including CMT type 2S (CMT2S), are inherited in an autosomal recessive pattern. As such, symptoms associated with CMT2S are typically less severe than in autosomal dominant forms of CMT2. The mutation associated with CMT2S is a mutation of the IGHMBP2 (immunoglobulin mu DNA binding protein 2) gene on chromosome 11, leading to abnormal RNA processing and axonal neuropathy.

### SUMMARY

An “antisense oligonucleotide” or “ASO” refers to a synthetic, multi-nucleotide RNA compound that hybridizes to a target RNA sequence in a manner such that gene expression can be inhibited, including by inactivating an mRNA molecule.

The invention provides a method of treating a patient diagnosed with MDS which comprises administering to the patient an amount of an ASO-T-JAK2 compound effective to treat MDS. In accordance with the present invention an ASO-T-JAK2 compound refers to any antisense oligonucleotide (ASO) targeted to a nucleic acid molecule encoding JAK2. In addition, an amount of the ASO-T-JAK2 compound effective to treat MDS requires administration of the compound in a quantity effective to inhibit JAK2 protein expression on the patient.

As noted above, the term “ASO-T-JAK2” refers to an ASO targeted to a nucleic acid molecule encoding the gene JAK2. The term “nucleic acid molecule” is a reference to a polynucleotide compound, i.e., RNA. An “antisense oligonucleotide” or “ASO” references a synthetic, multi-nucleotide DNA compound that hybridizes to a target RNA in a manner such that gene expression can be inhibited, including by inactivating an mRNA compound. Illustrative ASO-T-JAK2 compounds are disclosed herein (SEQ. ID. 4 and 5 targeting SEQ. ID. 2 and 3, respectively). Additional ASO-T-JAK2 compounds can be identified and prepared by methods known in the art for developing antisense oligonucleotides specific to a target RNA sequence.

The term “ASO-T-IGHMBP2” refers to an ASO targeted to a nucleic acid molecule encoding the IGHMBP2 gene. The term “nucleic acid molecule” is a reference to a polynucleotide compound, i.e., RNA. Illustrative ASO-T-IGHMBP2 compounds are disclosed and described in detail below. Additional ASO-T-IGHMBP2 compounds can be identified and prepared by methods known in the art for developing ASOs specific to a target RNA sequence.

A “patient” as referenced above references an individual diagnosed with, suffering from, or at risk of developing, being diagnosed with, or suffering from a disease or disorder, specifically PV or MDS or CMT2S.

In another embodiment, the invention provides a method of treating a patient diagnosed with PV which comprises administering to the patient an amount of an ASO-T-JAK2 compound effective to treat PV. An amount effective of the ASO-T-JAK2 compound effective to treat PV is an amount effective to inhibit JAK2 protein expression in the patient.

In yet another embodiment, the invention provides a method of inhibiting expression of the JAK2 gene in an individual which comprises administering to the individual an amount of an ASO-T-JAK2 compound effective to accomplish such inhibition.

In still another embodiment, the invention provides novel ASO-T-JAK2 compounds. As noted above, such compounds included, but are not limited to the oligonucleotides of SEQ ID 4 and SEQ ID 5. Numerous methods exist in the art for preparing ASOs once the nucleotide base sequence thereof have been determined. The determination of the appropriate sequence of bases for an ASO-T-JAK2 compound is likewise accomplished by strategies known in the art for identifying gene sequences for which an ASO would inhibit protein expression by the gene upon RNA binding.

In the treatment of an individual patient by administration of an ASO-T-JAK2, the dose and dosage regiment can be readily determined. Numerous examples of approved dosing for ASOs are known in the art, including nusinersen (marketed as Spinraza), mipomersen (marketed as Kynamro), and fomivirsen (marketed as Vitravene). In certain circumstances, it may be advantageous to administer the ASO-T-JAK2 in a derivatized or modified form in order to optimize bioavailability, duration of action, or therapeutic effect otherwise. Methods are known in the art for such modification/derivatization. See, generally, Drug Delivery Trends in Clinical Trails and Translational Medicine: challenges and opportunities in the delivery of nucleic acid-based therapeutics, J Pharm Sci. (2011 January); 100 (1): 38-52. For the purposes of the present invention the ASOs are administered intravenously, though other routes of administration are possible, including oral administration. Such alternative routes of administration are applicable to the ASOs described herein.

Accordingly, a further aspect of the present invention are pharmaceutical compositions that are used for the administration of the ASO-T-JAK2 compound to the patient being treated. These include sterile parenteral dosage forms of the type conventionally employed for the intravenous administration of a medicine. Such pharmaceutical compositions contain excipients that may be required. These can include one or more diluents, carriers, adjuvants, or combinations thereof as are known in the art for the production of a finished dosage form.

In one embodiment, the invention provides a method of treating a patient diagnosed with Charcot-Marie-Tooth type 2 (CMT2) which comprises: administering to said patient an amount of an ASO-T-IGHMBP2 compound effective to treat such disease.

In another embodiment, the invention provides a method of inhibiting expression of a mutated IGHMBP2 gene in an individual carrying a C31401A mutation of the IGHMBP2 gene, the method comprising: administering to the individual an amount of an ASO-T-IGHMBP2 compound effective to inhibit expression of the mutant IGHMBP2 gene in the individual.

In still another embodiment, the invention provides an antisense oligonucleotide (ASO) targeted to a nucleic acid molecule encoding a mutated IGHMBP2 gene. Such an ASO may be included in a pharmaceutical composition in combination with a pharmaceutically acceptable diluent, carrier, adjuvant, or combination thereof.

Numerous methods exist in the art for preparing ASOs once the nucleotide base sequence thereof have been determined. The determination of the appropriate sequence of bases for an ASO-T-IGHMBP2 compound is likewise accomplished by strategies known in the art for identifying gene sequences for which an ASO would inhibit protein expression by the gene upon RNA binding.

In the treatment of an individual patient by administration of an ASO-T-IGHMBP2, the dose and dosage regiment can be readily determined. Numerous examples of approved dosing for ASOs are known in the art, including nusinersen (marketed as Spinraza), mipomersen (marketed as Kynamro), and fomivirsen (marketed as Vitravene). In certain circumstances, it may be advantageous to administer the ASO-T-IGHMBP2 in a derivatized or modified form in order to optimize bioavailability, duration of action, or therapeutic effect otherwise. Methods are known in the art for such modification derivatization. See, generally, Drug Delivery Trends in Clinical Trails and Translational Medicine: challenges and opportunities in the delivery of nucleic acid-based therapeutics, J Pharm Sci. (2011 January); 100 (1): 38-52. For the purposes of the present invention the ASOs are administered intravenously, though other routes of administration are possible, including oral administration. Such alternative routes of administration are applicable to the ASOs described herein.

Accordingly, a further aspect of the present invention are pharmaceutical compositions that are used for the administration of the ASO-T-IGHMBP2 compound to the patient being treated. These include sterile parenteral dosage forms of the type conventionally employed for the intravenous administration of a medicine. Such pharmaceutical compositions contain excipients that may be required.

These can include one or more diluents, carriers, adjuvants, or combinations thereof as are known in the art for the production of a finished dosage form.

### BRIEF DESCRIPTION OF THE DRAWINGS

These and other features of this invention will be more readily understood from the following detailed description of the various aspects of the invention taken in conjunction with the accompanying drawings that depict various embodiments of the invention, in which:

FIG. 1 shows the results of a bioluminescence assay in which ASO treatment inhibits JAK2 expression and decreases cell viability;

FIGS. 2A and 2B show, respectively, fluorescent and brightfield images of SET-2 cells treated with ASOs according to embodiments of the invention;

FIGS. 3A and 3B show, respectively, fluorescent and brightfield images of HEL cells treated with ASOs according to embodiments of the invention;

FIGS. 4A and 4B show, respectively, fluorescent and brightfield images of SET-2 cells treated with ASOs according to embodiments of the invention; and

FIGS. 5A and 5B show, respectively, fluorescent and brightfield images of HEL cells treated with ASOs according to embodiments of the invention.

It is noted that the drawings of the invention are not to scale. The drawings are intended to depict only typical aspects of the invention, and therefore should not be considered as limiting the scope of the invention. In the drawings, like numbering represents like elements among the drawings.

### DETAILED DESCRIPTION

The present invention provides target sequences in the JAK2 pre-mRNA that are amenable to binding by a synthetic ASO. When so bound, expression of the JAK2 gene is inhibited. In patients diagnosed with MDS and PV, such inhibition of JAK2 expression provides an effective method of treatment of the disorder.

As noted above, MDS and PV are both associated with the V617F mutation of the JAK2 protein. The wildtype JAK2 protein sequence is shown in SEQ ID NO 1 in which the amino acid at position 617 is valine.

In one aspect of the present invention, a target region within the JAK2 pre-mRNA molecule is identified that, when bound with an ASO, is capable of inhibiting synthesis of the JAK2 protein. This region includes a 19 bp sequence:

(SEQ ID NO 2)
TTTCCTTAGTCTTTCTTTG

and the shorter 16 bp sequence:

(SEQ ID NO 3)
CCTTAGTCTTTCTTTG

Complementary ASO sequences are, respectively:

(SEQ ID NO 4)
5′-CAAAGAAAGACUAAGGAAA-3′
and:
(SEQ ID NO 5)
5′-CAAAGAAAGACUAAGG-3′

Each of these sequences bridges an intron and exon of the JAK2 pre-mRNA. When ASOs containing these sequences are bound to JAK2 pre-mRNA, processing of the pre-mRNA into mature mRNA is prevented, as is synthesis of the JAK2 protein.

To test the efficacy of ASOs having SEQ ID NO 4 and/or SEQ ID NO 5 to bind to the JAK2 pre-mRNA and inhibit JAK2 protein synthesis, three human cell lines (SET-2, HEL, CMK) are incubated with each of these ASOs and treated with cycloheximide. After a 48-hour incubation, the treated cell lines are collected and subjected to a CellTiter-Glo® (CTG) assay and RNA extraction. Data related to the cell lines and culture media are shown in Table 1 below.

TABLE 1

| Cell | | | |
|---|---|---|---|
| Line | Growth Period | | Culture Medium |
| SET-2 | 60-80 | hours | 80% RPMI 1640 + 20% heat-inactivated FBS |
| HEL | 36 | hours | 90% RPMI 1640 + 10% heat-inactivated FBS |
| CMK | 40-50 | hours | 80% RPMI 1640 + 20% heat-inactivated FBS |

Cells are harvested during the growth period and counted using a Cellometer K2. One million cells are incubated in the listed medium in each well of a six-well plate and incubated in a humidified incubator at 37° C. with 5% CO₂.

After 24 hours, the medium is changed, the ASO is added, and incubation is continued under the same conditions for another 24 hours. Cycloheximide is then added to a final concentration of 0.1 mg/mL. Twenty-four hours after addition of the cycloheximide, the medium and cells are combined in a 15 mL conical tube and 50 μL aliquots of cells are added to a 96-well assay plate for CTG assaying. Luminescence is recorded using a BioTek Synergy neo2 Multi-mode Reader. The remaining cells are spun at 4° C. to collect a cell pellet for RNA extraction by Tryzol reagent.

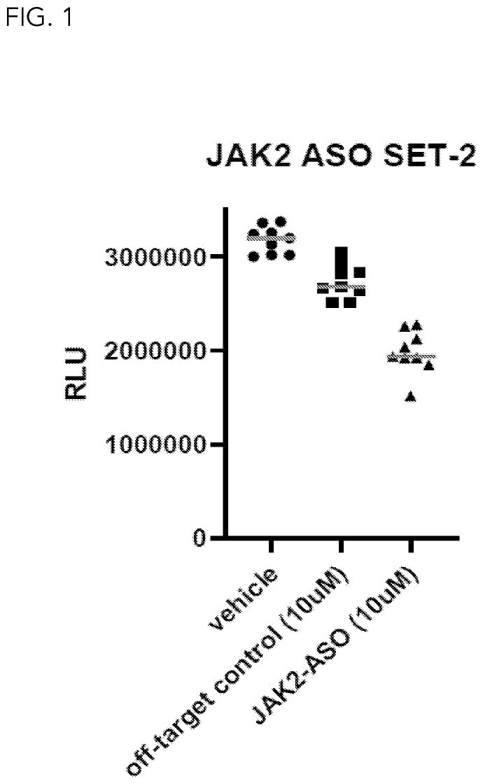

FIG. 1 shows results of the CTG assay described above for the SET-2 cell line. In the first column is the result for the assay vehicle, which contains no ASOs. Average relative luminescence units (RLUs) are in excess of 3,000,000. RLU is proportional to the number of viable cells in the assay well.

The second column shows the results of the assay when off-target control ASOs (i.e., ASOs not targeting JAK2 pre-mRNA or mRNA) are used. Average RLUs are reduced as compared to vehicle, but are still in excess of 2,500,000.

The third column of FIG. 1 shows the results of the assay when ASOs containing SEQ ID NO 4 or SEQ ID NO 5 are added. Here, average RLUs are reduced to less than 2,000,000, with some samples exhibiting a reduction to approximately 1,500,000. This constitutes a significant reduction in RLUs, and consequently cell viability, as compared to vehicle.

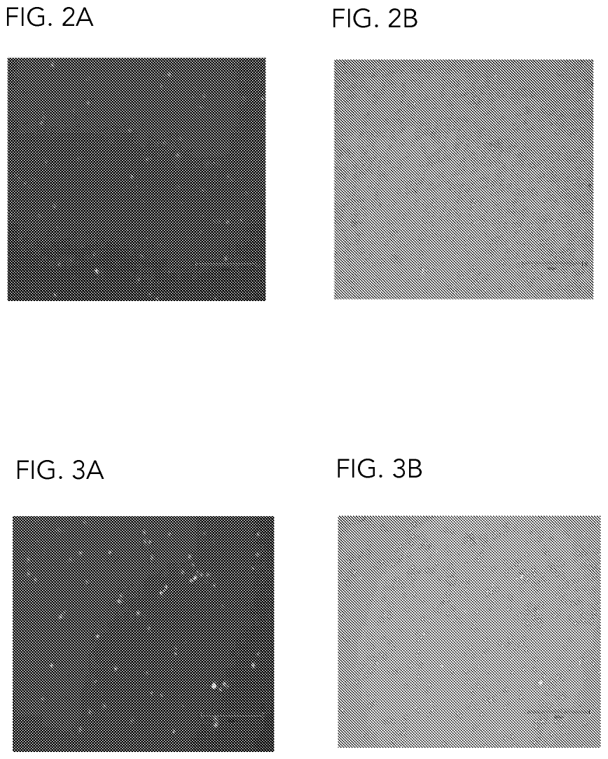

These results are confirmed with RNA sequencing using next-generation sequencing (NGS) to quantify expression of the JAK2 gene. FIGS. 2A and 2B show, respectively, fluorescent (GFP) and brightfield images of assay wells for SET-2 cells treated with ASOs containing SEQ ID NO 4 or SEQ ID NO 5. FIGS. 3A and 3B show, respectively, fluorescent and brightfield images for HEL cells treated with the same ASOs. In each case, fluorescence is lower than in untreated cells, demonstrating decreased mature JAK2 mRNA in samples incubated with ASOs containing SEQ ID NO 4 or SEQ ID NO 5.

Thus, embodiments of the invention include the administration of an ASO containing SEQ ID NO 4, SEQ ID NO 5, or both, to an individual in order to inhibit expression of JAK2; such administration as a treatment for MDS, PV, or both; ASOs targeting SEQ ID NO 2, SEQ ID NO 3, or both; ASOs having nucleotide sequences that include SEQ ID NO 4 or SEQ ID NO 5; and compositions containing such ASOs in combination with pharmaceutically acceptable diluents, carriers, adjuvants, or combinations thereof.

In other aspects, the present invention provides target sequences in the mutant IGHMBP2 pre-mRNA that are amenable to binding by a synthetic ASO. When so bound, expression of the mutant IGHMBP2 gene is inhibited. In patients diagnosed with CMT2S who are heterozygous for the mutation, such inhibition may result in the “rescue” or return to normal function of the IGHM1BP2 protein.

The full sequence of the wildtype IGHMBP2 gene is shown in the attached sequence listing as SEQ ID NO 6. The nucleotide at position 31401 is cytosine. In the mutation associated with CMT2S, this nucleotide is adenine.

In one aspect of the present invention, a target region within the resulting mutant IGHMBP2 pre-mRNA molecule is identified that, when bound with an ASO, is capable of inhibiting synthesis of the mutant IGHMBP2 protein. That region within the mutant IGHMBP2 gene comprises a 19-bp sequence that includes the C31401A mutation:

(SEQ ID NO 7)
CACTTCCACAGGGGGAAGA.

The complementary ASO sequence is:

(SEQ ID NO 8)
UCUUCCCCCUGUGGAAGUG.

An ASO comprising SEQ ID NO 3 bridges an intron and exon of the mutant IGHMBP2 pre-mRNA and, when bound to the pre-mRNA, prevents synthesis of the mutant IGHMBP2 protein.

To test the efficacy of an ASO comprising SEQ ID NO 8 in binding to the mutant IGHMBP2 pre-mRNA and inhibiting mutant IGHMBP2 protein synthesis, a fibroblast cell line of a patient with CMT2S is incubated with an ASO including SEQ ID NO 8 in RPMI 1640 medium supplemented with 10% FBS and treated with cycloheximide. After a 48-hour incubation, the treated cells are collected and for RNA extraction and Western Blot analysis to determine IGHMBP2 protein expression.

More specifically, one million cells are incubated in 2 mL in the RPMI 1640 medium described above in a humidified incubator at 37° C. with 5% carbon dioxide. After 24 hours, the medium is changed, the ASO is added, and incubation is continued for another 24 hours. Cycloheximide is then added to a final concentration of 0.1 mg/mL. Twenty-four hours after addition of the cycloheximide, the medium and cells are combined in a 15 mL conical tube and spun to collect a cell pellet for RNA extraction by Tryzol reagent. RNA extraction is carried out using, for example, Qiagen's RNeasy Mini Kit. Western Blot analysis is then carried out to determine the concentration of wild type and mutant proteins in the samples.

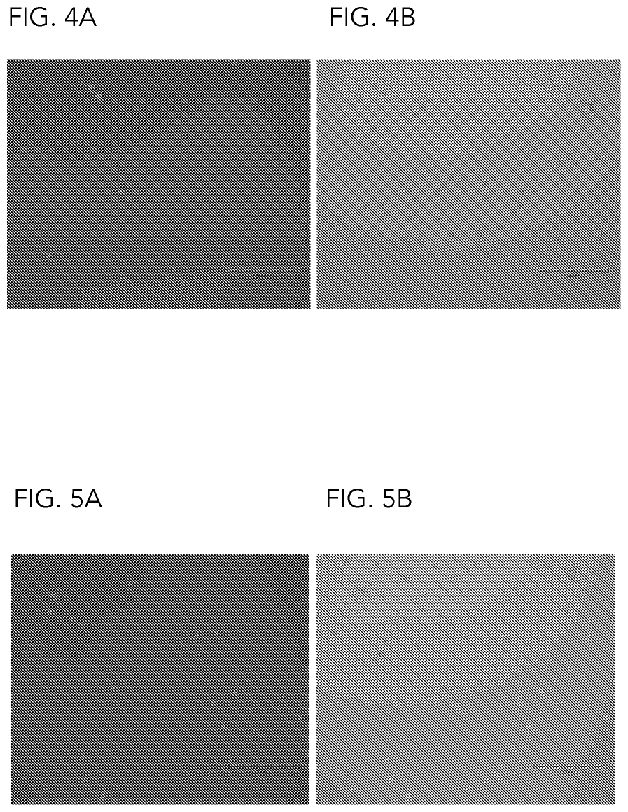

FIGS. 4A and 4B show fluorescent and brightfield photomicrographs, respectively, of SET-2 cells following treatment with ASOs including SEQ ID NO 8, demonstrating the ability of these ASOs to penetrate the cell membrane.

FIGS. 5A and 5B show similar fluorescent and brightfield photomicrographs, respectively, of HEL cells following treatment with ASOs including SEQ ID NO 8. Again, these results demonstrate the ability of such ASOs to penetrate the cell membrane.

ASOs other than or in addition to those including SEQ ID NO 8 may be useful in practicing the invention. Applicant has identified a “core” 11 nucleotide sequence within SEQ ID NO 8 that would provide a basis for sequences having sufficient specificity to enable discriminate binding of an ASO to the mutant IGHM1BP2 pre-mRNA. As one skilled in the art will understand, efficient and effective ASO therapies such as those described herein require the use of ASOs having a length sufficient to ensure discriminate binding of the ASO to the target sequence. ASOs including sequences of between 13 nucleotides and 35 nucleotides in length and including the core sequence of SEQ ID NO 9 are of sufficient length for use in practicing embodiments of the invention.

(SEQ ID NO 9)
5′-CCCCCUGUGGA-3′

Thus, an ASO having a nucleotide sequence that includes SEQ ID NO 4 and between two and 24 adjacent nucleotides are useful in practicing embodiments of the invention. Such nucleotide sequences include, for example, sequences as short as the 13-nucleotide sequences of SEQ ID NOS, 10, 11, and 12:

(SEQ ID NO 10)
5′-CCCCCUGUGGAAG-3′
(SEQ ID NO 11)
5′-UCCCCCUGUGGAA-3′
(SEQ ID NO 12)
5′-UUCCCCCUGUGGA-3′

Such nucleotide sequences also include, for example, sequences as long as the 35-nucleotide sequences of SEQ ID NOS 13 and 14.

(SEQ ID NO 13)
5′-GAAGAAAUCAAUCUUCCCCCUGUGGAAGUGAGGGC-3′
(SEQ ID NO 14)
5′-GAAAUCAAUCUUCCCCCUGUGGAAGUGAGGGCCAG-3′

More generally, ASOs having a nucleotide sequence between 13 nucleotides and 35 nucleotides in length comprising the core sequence of SEQ ID NO 9 and as many as 15 upstream nucleotides and/or 12 downstream nucleotides. The ASO “sequence window” is shown in SEQ ID NO 15.

(SEQ ID NO 15)
5′-GAAGAAAUCAAUCUUCCCCCUGUGGAAGUGAGGGCCAG-3′

As used herein, the singular forms “a,” “an,” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

This written description uses examples to disclose the invention, including the best mode, and also to enable any person skilled in the art to practice the invention, including making and using any devices or systems and performing any related or incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

1. A method of treating a patient diagnosed with myelodysplastic syndrome (MDS) or polycythemia vera (PV) which comprises:
administering to said patient an amount of an ASO-T-JAK2 compound effective to treat such disease, wherein the ASO-T-JAK2 compound is targeted to SEQ ID NO 2, SEQ ID NO 3, or both.

2. (canceled)

3. The method of claim 1, wherein the ASO-T-JAK2 compound comprises a nucleotide sequence including SEQ ID NO 4 or SEQ ID NO 5.

4. The method of claim 1, wherein the amount of said ASO-T-JAK2 compound is an amount sufficient to inhibit expression of JAK2 in the patient.

5-8. (canceled)

9. A method of inhibiting expression of the JAK2 gene in an individual which comprises:
administering to the individual an amount of an ASO-T-JAK2 compound effective to inhibit JAK2 expression in the individual, wherein the ASO-T-JAK2 compound is targeted to SEQ ID NO 2, SEQ ID NO 3, or both.

10. (canceled)

11. The method of claim 9, wherein the ASO-T-JAK2 compound comprises a nucleotide sequence including SEQ ID NO 4 or SEQ ID NO 5.

12. An antisense oligonucleotide (ASO) targeted to a nucleic acid molecule encoding JAK2, wherein the ASO-T-JAK2 compound is targeted to SEQ ID NO 2, SEQ ID NO 3, or both.

13. (canceled)

14. The ASO of claim 12, wherein the ASO comprises a nucleotide sequence selected from a group consisting of: SEQ ID No 4 and SEQ ID NO 5.

15. A pharmaceutical composition which comprises:
the ASO of claim 12; and
a pharmaceutically acceptable diluent, carrier, adjuvant, or combination thereof.

16. The composition of claim 15 which further comprises a sterile parenteral dosage form.

17. A method of treating a patient diagnosed with Charcot-Marie-Tooth type 2 (CMT2) which comprises:
administering to said patient an amount of an ASO-T-IGHMBP2 compound effective to treat such disease.

18. The method of claim 17, wherein the patient is diagnosed with Charcot-Marie-Tooth type 2S (CMT2S).

19. The method of claim 18, wherein the ASO-T-IGHMBP2 compound is targeted to SEQ ID NO 7.

20. The method of claim 19, wherein the ASO-T-IGHMBP2 compound comprises a nucleotide sequence including SEQ ID NO 9.

21. The method of claim 20, wherein the ASO-T-IGHMBP2 compound comprises a nucleotide sequence including SEQ ID NO 8.

22. The method of claim 20, wherein the ASO-T-IGHMBP2 compound comprises a nucleotide sequence selected from a group consisting of: SEQ ID NO 10, SEQ ID NO 11, and SEQ ID NO 12.

23. The method of claim 20, wherein the ASO-T-IGHMBP2 compound comprises a nucleotide sequence 13 to 35 nucleotides in length within SEQ ID NO 15.

24-37. (canceled)
